(19) United States
(12) Patent Application Publication
(10) Pub. No.: US 2004/0148016 A1
(43) Pub. Date: Jul. 29, 2004

(54) Biocompatible medical device coatings

(76) Inventors: Dean A. Klein, North Oaks, MN (US); James D. Brazil, Braham, MN (US)

Correspondence Address: FAEGRE & BENSON LLP, 2200 WELLS FARGO CENTER, 90 SOUTH 7TH STREET, MINNEAPOLIS, MN, 55402, US

(21) Appl. No.: 10/702,846

(22) Filed: Nov. 6, 2003

Related U.S. Application Data
(60) Provisional application No. 60/424,559, filed on Nov. 7, 2002.
(60) Provisional application No. 60/427,270, filed on Nov. 18, 2002.

(51) Int. Cl. A61F 2/06

(52) U.S. Cl. 623/1.42

(57) ABSTRACT

The present invention provides a coated medical device including a biocompatible coating. The biocompatible coating includes a phenolic resin and at least one particulate material dispersed within the resin that affects a functional property of the coating. For example, the particulate material may affect surface properties of the coating, such as lubricity, sorption and surface area. The biocompatible coating of the present invention is biocompatible and adheres well to a variety of medical devices. The biocompatible coating may be enhanced by the incorporation of lubricants or bioactive agents into or onto the coating.

### CROSS-REFERENCE TO RELATED APPLICATION

This application claims the benefit of U.S. Provisional Patent Application Serial Nos. 60/424,559 having a filing date of Nov. 7, 2002, and 60/427,270 having a filing date of Nov. 18, 2002, both entitled “Stent Coatings.” Both of these applications are incorporated herein by reference.

### BACKGROUND OF THE INVENTION

Biocompatible coatings for implantable medical devices are becoming increasingly important throughout the medical device industry. For example, a significant amount of effort has been invested in developing biocompatible coatings for stents. Stents are typically implanted within an internal lumen in a contracted state and expanded when in place in the lumen to maintain the patency of the lumen and to allow fluid flow through the lumen. Stents may be delivered to a desired lumen site in a number of ways, such as by placing the stent on a balloon portion of a catheter, positioning the catheter in the lumen and then expanding the stent by inflation of the balloon. The stent may then be left in place by deflating the balloon and removing the catheter.

Metal-containing medical devices are often preferred over other types of medical devices because the metallic structure provides the durability and strength required for a variety of medical device applications. However, bare metal medical devices have several drawbacks. First, such medical devices may be difficult to deliver to an anatomical site in a patient due to friction between the medical device and the patient's anatomy. This friction may damage the anatomy, further complicating the medical device delivery process. Additionally, such medical devices may not be biocompatible with the body. This may result in adverse reactions with the patient's anatomy. Further yet, such medical devices may not be effectively suitable for releasing bioactive agents directly into the anatomical site to be treated.

To overcome these deficiencies, various medical device coatings have been employed. For example, lubricious coatings such as polyvinylpyrrolidone (PVP), polyurethane, polyester, vinyl resin, fluorocarbons, silicone, rubber and combinations thereof have been applied to medical devices such as stents. Hydrophilic coatings containing PVP and cellulose ester polymers as reported in U.S. Pat. Nos. 5,001,009 and 5,525,348 to Whitbourne have also been applied to stents.

Unfortunately, many of these coated medical devices also suffer from drawbacks. For example, these reported coatings may suffer from poor adhesion to medical device surfaces, poor lubricity, poor drug releasing properties and/or poor biocompatibility. Thus, there is a need in the art for a medical device coating, more particularly, a stent coating, that is lubricious, biocompatible and/or capable of releasing active ingredients directly to a lumen site.

### SUMMARY OF THE INVENTION

In one embodiment, the present invention provides a coated medical device including a substrate and biocompatible coating fixed to a surface of the substrate. The biocompatible coating is composed of a phenolic binder or resin (collectively referred to herein as “resin”), and at least one particulate material dispersed therein. As used herein, the phrases “dispersed” and/or “impregnated” refer both to particles located within the phenolic resin and particles located at an exposed surface of the phenolic resin.

The phenolic resin may include a phenol-aldehyde resin or a polyvinyl phenol resin. The particulate material may affect a functional property of the coating. For example, the particulate material may affect the lubricity, sorption, surface area, adhesion, radiopacity, durability or the controlled release capability of the coating. The particulate material may include, for example, a metallic, polymeric or ceramic material. Suitable metallic materials may contain transition metals such as molybdenum. Suitable polymeric materials include polytetrafluoroethylene (PTFE) and polyurethane. In a particular embodiment, the first particulate material is composed of molybdenum disulfide. The particles may have a major dimension that is less than, greater than or equal to the thickness of the coating in which the particles are dispersed. In one embodiment, for example, the particles may have a major dimension of less than about 300 mil, more particularly less than about 200 mil, even more particularly less than about 100 mil. In an alternate embodiment, the particles may have a major dimension of less that about 3 mil, more particularly, less than about 2 mil, even more particularly, less than about 1 mil.

In another embodiment, the biocompatible coating of the present invention is composed of a first and a second particulate material. The second particulate material may also affect a functional property of the coating such as the lubricity, sorption or surface area of the coating. The second particulate material may also be a metal, a polymer or a ceramic. In one embodiment, the first particulate material is composed of molybdenum disulfide and the second particulate material is composed of a polymer. More than two particulate materials may be added as desired to further affect the functional properties of the coating.

In yet another embodiment, the biocompatible coating includes a bioactive agent applied onto, or dispersed within, the resin. Any suitable bioactive agent may be incorporated into the coating including both natural and synthetic agents. Suitable bioactive agents include, for example, antiplatelets, antithrombins, cytostatic agents, antiproliferative agents, vasodilators, antimicrobials, antibiotics, antimitotics, antisecretory agents, non-steroid anti-inflammatory agents, immunosuppressive agents, growth factor antagonists, free radical scavengers, antioxidants, radiotherapeutic agents, radiopaque agents, radiolabeled agents, nucleotides, cells, proteins, glycoproteins, isolates, enzymes, hemostatic agents, ribonucleases and combinations of these bioactives. The coating may further include a bioabsorbable lubricious material applied onto a surface of the biocompatible coating. The lubricious material may be a naturally-derived biocompatible compound such as β-glucan.

In another embodiment, the present invention provides a coated stent including a stent body and biocompatible coating fixed to the stent body. The biocompatible coating is composed of a phenolic resin and at least one particulate material dispersed therein.

In a further embodiment, the present invention provides a method of treating stenosis by inserting the coated stent of embodiments of the present invention into a lumen while the stent is in a contracted state, and then expanding the stent once in the lumen.

### BRIEF DESCRIPTION OF THE DRAWINGS

FIG. 1 is a microscopic image of a coated stent at 50× magnification according to an embodiment of the present invention.

FIG. 2 is a microscopic image of a coated stent at 50× magnification according to another embodiment of the present invention.

FIG. 3 is a microscopic image of a coated stent at 200× magnification according to a further embodiment of the present invention.

FIG. 4 is a microscopic image of a coated stent at 200× magnification according to another embodiment of the present invention.

FIG. 5 is a microscopic image of a biocompatible coating according to yet another embodiment of the present invention.

### DETAILED DESCRIPTION OF THE INVENTION

The present invention provides a biocompatible coating or outer layer for a medical device. The coating is generally composed of a phenolic resin applied onto a medical device. The resin is impregnated with a first particulate material that may affect a functional property of the coating. Optionally, the coating may include at least two particulate materials dispersed within the resin that also affect one or more functional properties of the coating.

The biocompatible coatings of the present invention may be applied onto any suitable medical device. For example, the biocompatible coatings may be applied to stents, artificial joints, plates, screws, pins, markers, leads, catheters, electronic devices such as pacemakers, and other various load bearing and non-load bearing devices. The medical device may be formed from, for example, metallic, polymeric, ceramic, carbon or silicon materials. Suitable metals include stainless steel, nickel, titanium, tantalum, platinum, cobalt, chromium, nitinol and combinations or alloys of these materials. Suitable polymeric materials may include thermoset or thermoplastic polymers, including polyurethane, polypropylene, polyethylene and other suitable polymers. Optionally, prior to application of the biocompatible coating, the medical device surface may be treated to improve the surface properties of the medical device. Suitable treatments may include texturing, anodizing and/or graining the medical device surface.

The phenolic resin of the present invention may be composed of any suitable biocompatible phenolic material that adheres or bonds to the medical device and is substantially biocompatible. Phenolic resins are thermosetting resins obtained by the condensation of phenol or substituted phenols with aldehydes. Suitable phenolic resins may include biocompatible phenol-aldehyde resins such as one-stage and two-stage phenol-formaldehyde resins, as well as polyvinyl phenol resins. Suitable one and two-stage phenol formaldehyde resins include resole and novolak resins. The phenolic resin may also be modified by incorporating cresols, resorcinol and/or furfural.

Examples of suitable novolak resins include novolaks obtained by polycondensing at least one kind of aromatic hydrocarbon such as phenol, m-cresol, o-cresol, p-cresol, 2,5-xylenol, 3,5-xylenol, resorcin, pyrogallol, bisphenol, bisphenol A, trisphenol, o-ethylphenol, m-ethylphenol, p-ethylphenol, propyl phenol, n-buylphenol, t-butylphenol, t-butylphenol, 1-naphthol and 2-naphthol with at least one aldehyde (e.g. formaldehyde, acetaldehyde, propionaldehyde, benzaldehyde and furfural) or ketone (e.g. acetone, methyl ethyl ketone and methyl isobutyl ketone) in the presence of an acidic catalyst. Paraformaldehyde and paraaldehyde may be respectively used in place of formaldehyde and acetaldehyde.

In one embodiment, the aromatic hydrocarbons of the novolak resin are obtained by polycondensing at least one kind of phenol selected from phenol, m-cresol, o-cresol, p-cresol, 2,5-xylenol, 3,5-xylenol and resorcin with at least one kind of aldehyde selected from formaldehyde, acetaldehyde and propionaldehyde.

In certain embodiments, the novolak resins may be polycondensates of phenols and aldehydes in an approximate mixing molar ratio of, for example, m-cresol:p-cresol:2,5-xylenol:3,5-xylenol:resorcin=40-100:0-50:0-20:0-20:0-20. Alternatively, the novolak resins may be polycondensates of phenols and aldehydes in an approximate mixing molar ratio of phenol:m-cresol:p-cresol=70-100:0-30:0-20. Further yet, the novolak resins may be polycondensates of phenols and aldehydes in an approximate mixing molar ratio of phenol:m-cresol:p-cresol=10-100:0-60:0-40. In yet a further embodiment, the weight-average molecular weight (relative to polystyrene standards) as measured by gel permeation chromatography of the novolak resin may be between about 500 to about 30,000.

Examples of polyvinyl phenolic resins include o-hydroxystyrene, m-hydroxystyrene, p-hydroxystyrene, 2-(o-hydroxyphenyl)propylene, 2-(m-hydroxyphenyl)propylene and 2-(p-hydroxyphenyl)propylene, and combinations, derivative or copolymers thereof. The hydroxystyrenes may have a halogen such as chlorine, bromine, iodine or fluorine, or a C₁-C₄ alkyl substituent in the aromatic ring.

The polyvinyl phenolic resin is usually synthesized by radical polymerization or cationic polymerization of one or more hydroxystyrenes. Such a polyvinyl phenolic resin may be partially hydrogenated. It may also be a resin wherein OH groups of polyvinyl phenols are protected with a t-butoxycarbonyl group, a pyranyl group or a furanyl group. The weight-average molecular weight of the polyvinyl phenol resin may be within a range from 1,000 to 100,000.

The phenolic resins of the present invention may possess several characteristics favorable for application to stents. First, phenolic resins may provide a strong bond or adhesion to medical devices without requiring pre-treatment of the medical device surface. Further, suitable phenolic resins may be biocompatible and suitable for permanent insertion into a body lumen. Further yet, suitable phenolic resins have a microscopic surface topology that may provide enhanced surface properties for application of a lubricant or bioactive agent. The particular phenolic resin employed will depend on the medical device being coated and the specific applications of that medical device. Phenolic resins suitable for use in embodiments of the present invention may be available from, for example, Plenco, Sheboygan, Wis. and Tipco Industries Limited, Mumbai, India.

The phenolic resin of the present invention may be impregnated with at least one particulate material to affect one or more functional properties of the phenolic resin as desired. In one embodiment, the particulate material may increase the lubricity of the coating. This may provide for easier stent delivery to a desired site within a body lumen. In another embodiment, the particulate material may increase the sorption of bioactive agents and/or lubricants. In yet another embodiment, the particulate material may affect the surface topology of the phenolic resin. In a further embodiment, the particle may enhance the ability of the coating to provide controlled or sustained release of a biologically active agent. The enhanced surface topology may help maintain the stent in a desired lumen location, as well as providing for greater surface area to accept a lubricant, bioactive agent or other desired material. Additional functional properties that may be affected by the particles include coating adhesion, radiopacity and durability. Examples of suitable particulate materials include metallic, polymeric and ceramic materials. For example, suitable metallic materials include transition metals such as molybdenum, more particularly, molybdenum disulfide. Examples of suitable polymers include polyurethanes and fluorocarbons such as polytetrafluoroethylene (PTFE). Examples of suitable ceramics include zirconium oxide and aluminum oxide.

In yet another embodiment, two or more particulate materials may be dispersed within the resin to affect one or more functional properties of the coating. For example, one of the particulate materials could be selected to affect the sorption of the coating, and the second material could affect the lubricity of the coating. In another example, both particulate materials could affect the sorption of the material in varying degrees to provide for differing rates of bioactive agent release. In a specific embodiment, the biocompatible coating may be composed of a phenolic resin impregnated with molybdenum disulphide particles and polymeric particles. In yet another embodiment, more than two particulate materials may be dispersed within the resin as desired. In an alternate embodiment, the particles reported herein may disposed on an exposed surface of the phenolic resin.

The particles may be sized to have a major dimension less than, greater than or equal to the thickness of the biocompatible coating. For example, the particles may have a major dimension of less than 300 mil, more particularly, less than about 200 mil, even more particularly, less than about 100 mil. In an alternate embodiment, the particles may have a major dimension of less than about 3 mil, more particularly, less than about 2 mil, even more particularly, less than about 1 mil.

Suitable phenolic coatings having particles dispersed therein are available, for example, from KG industries, Inc., Hayward, Wis. (e.g., Gun Kote brand coating, catalog #2401), EM Coatings, Peachtree City, Ga. (e.g. Everlube brand coating, catalog #6102-G), and E.I. Dupont & Nemours (e.g., Teflon-S One Coat brand coating).

The coating of the present invention may be applied onto a suitable medical device in a variety of ways. For example, the coating may be dissolved or dispersed in a suitable organic solvent or aqueous solution to form a coating material, which may then be applied to the medical device by various techniques such as by dipping, pouring, pumping, spraying, brushing, wiping or other known methods. Suitable organic solvents include cellosolve-based solvents, propylene glycol-based solvents, ester-based solvents, alcohol-based solvents, ketone-based solvents, and high polar solvents such as dimethylformamide, dimethylacetamide or N-methyl pyrrolidone. Other examples of suitable solvents include hexane, cyclohexane, trichloroethane, carbon tetrachloride, toluene, ethyl acetate, trichloroethylene, methyl ethyl ketone, cyclohexanone, methyl acetate, dioxane, acetone, carbon disulfide nitrobenzene, nitromethane, ethanol, dimethyl sulfoxide, ethylene carbonate, phenol and methanol. The coating material may further include suitable dispersing agents and other additives.

The particulate material may be combined with the phenolic resin either prior to or after coating the medical device. For example, the particulate material may be combined into the dissolved phenolic resin prior to applying the coating onto the medical device. Alternatively, the particles may be dispersed onto the surface of the resin prior to curing or drying the coating as described below. The amount or concentration of particles dispersed within or onto the resin will vary depending upon the medical device, and on the particular property that is desired for a given application. For example suitable particle concentration may range from between about 0.5 and about 75 weight percent.

After application onto the stent, the coating may then be bonded to the medical device by drying or curing the coating. For example, the coating may be air dried or thermally cured at between about 100° F. and about 500° F. In one embodiment, the coating may be cured by heating at about 325° F. In another embodiment, the curing process may include the use of a suitable catalyst such that curing occurs at below about 200° F. Alternatively, the coating may be bonded by exposure to UV radiation. Optionally, after curing, additional layers of the biocompatible coating may be applied to provide additional coating thickness. These multiple coating layers may be identical or varied in composition as desired for a particular application.

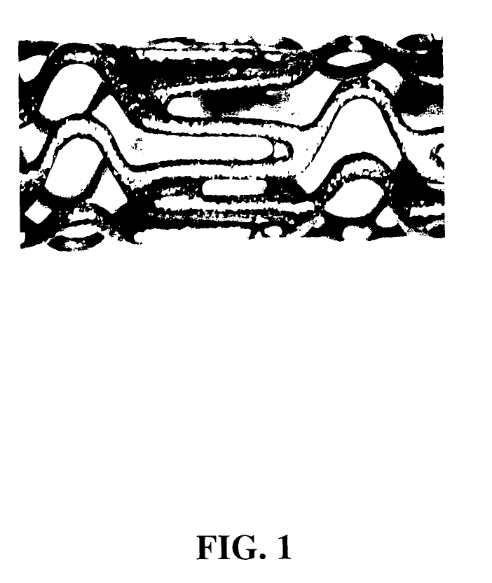

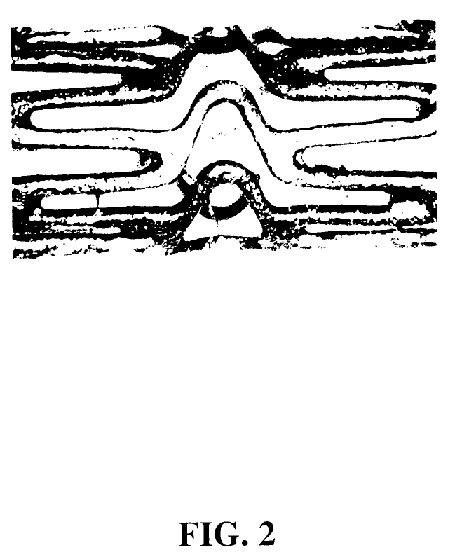

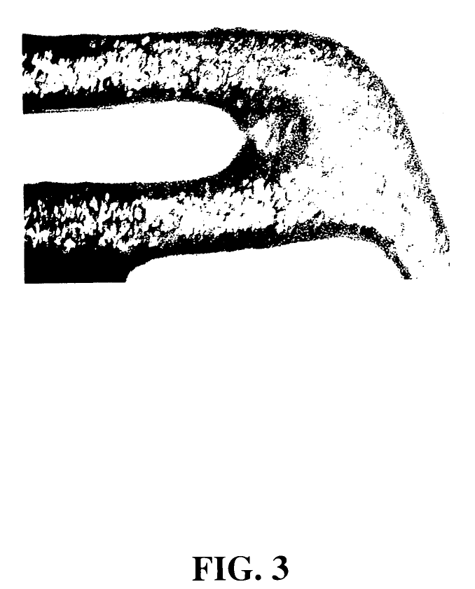

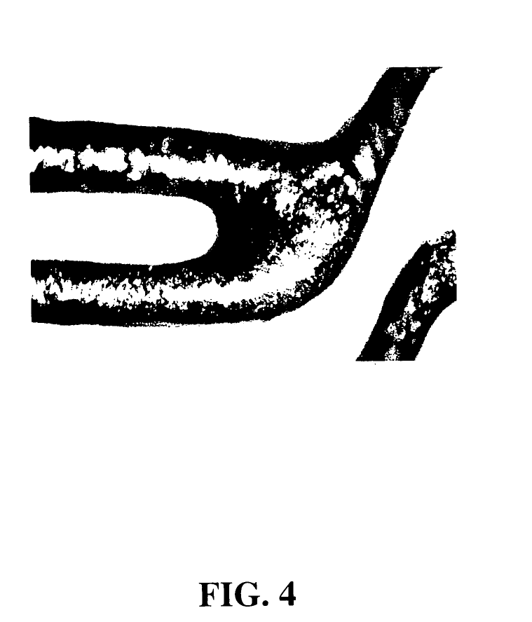

The resulting coating may be a partial or generally continuous coating over the surface of the medical device. For example, if the coating is applied onto a stent, the spaces formed within the stent pattern may or may not be filled by the coating. However, as illustrated in FIGS. 1-4, the various segments, bridges, or struts that form the stent pattern may be completely covered by the biocompatible coating of the present invention.

The thickness of the coating will vary depending on the desired application and the specific coating composition employed, and a broad range of coating thickness may be suitable for the present invention. In one embodiment, the average coating thickness may range from a molecular or nanolayer film to a coating of about 300 mil. In another embodiment, the coating thickness may range from a flash coating of about 1 mil to about 300 mil. In yet another embodiment, the coating thickness may range from about 2 mil to 200 mil. In a further embodiment, the coating thickness may range from about 3 mil to 100 mil. Generally, thinner coatings may result in less coating fatigue, particularly for dynamic medical devices such as expandable stents. Nonetheless, any fatigue caused during stent expansion and/or deformation should not adversely affect the functional properties of the coating.

The resulting biocompatible coating provides several beneficial characteristics. First, the coating is biocompatible and does not substantially degrade within the body. Additionally, the coating may be fixed to a variety of medical devices. Further, the coating is sufficiently durable to withstand the rigors of medical device implantation, and operation (e.g., stent expansion), without significant degradation. Further yet, the coating is sufficiently lubricious to provide for effective medical device delivery.

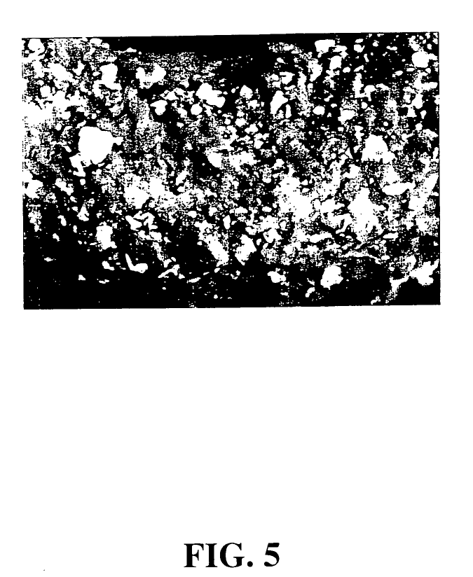

Additionally, the coating of the present invention may have a significant degree of microscopic topology. FIGS. 1-5 illustrate a stent coated with Gun Kote brand coating available from KG Industries, Hayward, Wis. The topology evident in these Figures generally results in a coating with an increased surface area. An increased surface area may provide several advantageous characteristics. First, an increased surface area may help maintain the stent at a desired location in a lumen, potentially resulting in greater effectiveness in treating a condition. Second, the surface area may provide for greater sorption of lubricants and bioactive agents that may be applied onto the coating. This sorption of lubricants and bioactive materials may be further enhanced by the presence of particulate materials at the surface of the coating. Third, the surface area may provide for enhanced release of bioactive agents after delivery to a anatomical site.

Optionally, the biocompatible coating of the present invention may include additional polymers, as well as additives as desired for a particular application. Examples of suitable additives may include dyes, pigments, surfactants, adhesives, catalysts, radiopaque materials and radiation absorptive materials.

In one embodiment of the present invention, the coating may include one or more bioactive agents applied to, or dispersed within, the phenolic resin. The bioactive compound may treat a variety of conditions, including restenosis, thrombosis, infection and inflammation. The bioactive agent may be any suitable natural or synthetic agent, including antiplatelets, antithrombins, cytostatic agents, antiproliferative agents, vasodilators, antimicrobials, antibiotics, antimitotics, antisecretory agents, non-steroid anti-inflammatory agents, immunosuppressive agents, growth factor antagonists, free radical scavengers, antioxidants, radiotherapeutic agents, radiopaque agents, radiolabelled agents, nucleotides, cells proteins, glycoproteins, isolates, hemostatic agents and ribonucleases. Examples of specific bioactive agents include heparin, hirudin, argatroban, forskolin, vapiprost, prostacyclin, dextran, D-phe-pro-arg-chloromethylketone, dipyridamole, glycoprotein antibody, recombinant hirudin, thrombin inhibitor, angiopeptin, angiotensin converting enzyme inhibitors, calcium channel blockers, colchicine, fibroblast growth factor antagonists, HMG-CoA reductase inhibitor, methotrexate, monoclonal antibodies, nitroprusside, phosphodiesterase inhibitors, prostaglandin inhibitor, seramin, serotonin blockers, steroids, thioprotease inhibitors, triazolopyrimidine and other PDFG antagonists, alpha-interferon and genetically engineered epithelial cells, dexamethasone derivatives and other anti-inflammatory steroids and combinations thereof.

In one embodiment, the bioactive agent is dispersed within the resin. The bioactive agent may be added to the resin prior to coating and/or curing. In these embodiments, the bioactive agent must be able to withstand the coating and/or curing process. In another embodiment, the bioactive agent is applied onto the cured coating of the present invention. In these embodiments, the increased surface area and composition of the coating may provide greater sorption of the active ingredient. This sorption may be enhanced by the particulate material dispersed within the phenolic resin. In yet another embodiment, the bioactive agent is disposed in between two or more coating layers.

There are several benefits to delivering bioactive agents according to the present invention. First, the coating of the present invention provides an optimal surface for sorption of the bioactive agent after bonding the coating to the medical device. This reduces the likelihood that the bioactive agent will be adversely affected during the medical device coating process. Further, direct delivery of the bioactive agent may be more effective than delivery by ingestion or injection because the bioactive agent may be delivered directly to the desired site without any degradation in vivo. Further yet, coatings of the present invention may control the rate of release of the bioactive agent.

In an alternate embodiment, the present invention may be composed of the biocompatible coating described herein and a lubricant applied onto the surface of the biocompatible coating. The lubricant may further decrease the coefficient of friction of the surface of the coating such that delivery of the medical device to an anatomical site is enhanced. Suitable lubricants are generally biocompatible and bioabsorbable, such that upon delivery of the medical device to a desired site, the lubricant is safely dispersed into the body and the position of the medical device may then be maintained. Suitable lubricants may contain naturally-derived materials such as β-glucan. β-glucan is a naturally occurring constituent of cell walls in essentially all living systems including plants, yeast, bacteria, and mammalian systems.

Aqueous solutions of β-glucan may be applied to the coated medical device in a conventional manner, and then dried to form a powder-like substance on the surface of the coating. Upon contact with body fluids at the anatomical site, the powdered β-glucan re-hydrates to facilitate delivery of the medical device to a desired location. The β-glucan solution has excellent lubricity, thereby providing an optimum lubricant for stent delivery. The re-hydrated β-glucan may then be absorbed by the body during and/or after delivery. Suitable β-glucan compositions include β-D-glucans containing 4-0-linked-β-D-glycopyranosyl units and 3-0-linked-β-D-glycopyranosyl units, or 5-0-linked-β-D-glycopyranosyl units and 3-0-linked-β-D-glycopyranosyl units.

In one embodiment, the coating may be applied to a stent, more particularly a vascular stent. The coated stent of the present invention may be used to treat stenosis of body lumens, in particular, stenosis of blood vessels. The stent may be delivered in a conventional manner, such as by attaching the coated stent to a catheter while in a contracted position, inserting the catheter into the lumen, expanding the stent to an expanded position, and then removing the catheter from the lumen.

What is claimed is:

1. A coated medical device comprising:
an implantable substrate; and
a biocompatible coating fixed to a surface of the substrate, the coating including a phenolic resin and at least one particulate material dispersed within the phenolic resin that affects a functional property of the coating.

2. The medical device of claim 1 wherein the phenolic resin comprises a resole resin, a polyvinyl phenol resin or a novolak resin.

3. The medical device of claim 1 wherein the particulate material comprises a polymeric material, a metal, a ceramic material or a combination thereof.

4. The medical device of claim 1 wherein the particulate material comprises a transition metal.

5. The medical device of claim 1 wherein the particulate material comprises molybdenum.

6. The medical device of claim 1 wherein the particulate material comprises molybdenum disulfide.

7. The medical device of claim 1 wherein the particulate material comprises a fluorocarbon.

8. The medical device of claim 1 wherein the particulate material comprises PTFE.

9. The medical device of claim 1 wherein the particulate material comprises zirconium oxide.

10. The medical device of claim 1 wherein the particles have a major dimension of less than 300 mil.

11. The medical device of claim 1 wherein the particles have a major dimension of less than about 3 mil.

12. The medical device of claim 1 comprising at least two particulate materials dispersed within the phenolic resin.

13. The medical device of claim 1 further comprising a biologically active agent.

14. The medical device of claim 13 wherein the biologically active agent is dispersed within the phenolic resin.

15. The medical device of claim 13 wherein the biologically active agent is applied onto an exposed surface of the phenolic resin.

16. The medical device of claim 13 wherein the biologically active agent comprises an anti-thrombin, anti-microbial, anti-restenosis or anti-inflammatory agent.

17. The medical device of claim 1 further comprising a lubricant applied onto an exposed surface of the phenolic resin.

18. The medical device of claim 17 wherein the lubricant comprises β-glucan.

19. The medical device of claim 1 comprising a plurality of coating layers.

20. The medical device of claim 1 comprising a first coating layer applied to the medical device, and a biologically active agent, lubricant, or both applied to an exposed surface of the first coating layer.

21. The medical device of claim 1 comprising a first coating layer applied to the surface of the medical device, a second coating layer applied to the first coating layer, and a biologically active agent disposed between the first and second layers.

22. The medical device of claim 1 wherein a surface of the medical device is coated with a biologically active agent prior to the application of the coating.

23. The medical device of claim 1 wherein the coating has a thickness of less than about 300 mil.

24. The medical device of claim 1 wherein the coating has a thickness of between about 1 mil and 300 mil.

25. The medical device of claim 1 wherein the coating has a thickness of between about 2 mil and 200 mil.

26. A coated stent comprising:
a stent body; and
a biocompatible coating fixed to the stent body, the coating including a phenolic resin and at least one particulate material dispersed within the phenolic resin that affects a functional property of the coating.

27. The coated stent of claim 26 wherein the stent body comprises a metal or polymeric material.

28. The coated stent of claim 26 wherein the stent body is self-expandable or balloon expandable.

29. A method of treating stenosis of a lumen comprising:
inserting into the lumen a coated stent in a contracted position, the coated stent comprising a stent body and a biocompatible coating fixed to the stent body, the coating including a phenolic resin and at least one particulate material dispersed within the phenolic resin that affects a functional property of the coating; and
expanding the stent into an expanded position within the lumen.

30. The method of claim 29 wherein the expanding step comprises self-expanding or balloon-expanding the stent from the contracted to the expanded position.
